(19) United States
(12) Patent Application Publication
(10) Pub. No.: US 2008/0108959 A1
(43) Pub. Date: May 8, 2008

(54) SOLID TYPE MICRONEEDLE AND METHODS FOR PREPARING IT

(75) Inventors: Hyung Il JUNG, Seoul (KR); Kwang Lee, Seoul (KR)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Industry-Academic Cooperation Foundation, Yonsei University, Seoul (KR)

(21) Appl. No.: 11/972,315

(22) Filed: Jan. 10, 2008

(30) Foreign Application Priority Data
Jul. 21, 2006 (KR) 10-2006-0068513
Jul. 20, 2007 (KR) PCT/KR2007/003506

(51) Int. Cl. A61M 5/32

(52) U.S. Cl. 604/272

(57) ABSTRACT

Disclosed herein are biodegradable solid microneedles and a fabrication method thereof. The microneedles are small in diameter and are long and hard enough to pass through the stratum corneum. Thus, the biodegradable solid microneedles can be used for painless transdermal drug delivery, the detection of biological samples such as blood, and biopsy.

### TECHNICAL FIELD

The present invention relates to solid microneedles and a fabrication method thereof. Furthermore, the present invention relates to in-vivo delivery of a drug or a cosmetic component through solid microneedles.

### BACKGROUND ART

Generally, microneedles are used in in-vivo drug delivery, the detection of biological samples, and biopsy. Drug delivery with microneedles aims to deliver a drug through the skin rather than biological circulatory systems such as blood vessels or lymphatic vessels. Accordingly, the microneedles should not cause pain when they penetrate the skin, and should have sufficient length such that they can deliver drugs to the target site. In addition, the microneedles should have excellent physical hardness such that they can penetrate the stratum corneum having a thickness of 10-20 μm. Since in-plane microneedles were suggested (“Silicon-processed Microneedles”, Journal of microelectrochemical systems Vol. 8, Nol, March 1999), various types of microneedles have been developed. For example, a solid silicon microneedle array fabricated using an etching method was suggested as an out-of-plane microneedle array (US Patent Publication No. 2002138049, entitled “Microneedle devices and methods of manufacture and use thereof”). However, the solid silicon microneedle according to this method has a diameter of 50-100 μm and a length of 500 μm, and thus it has problems that it is impossible to realize painless skin penetration and that in-vivo delivery of a drug or a cosmetic component to the target site is not reliably achieved. An array of transdermal microneedles was suggested by Nano-devices & systems Inc. (Japanese Patent Publication No. P2005-154321; and “Sugar Micro Needles as Transdermic Drug Delivery System”, Biomedical Microdevices 7:3, 185188, 2005). Such transdermal microneedles are used for drug delivery or cosmetic purposes and are not removed after their insertion into the skin. In this method, the microneedle array is fabricated by adding a composition, comprising a mixture of maltose and a drug, to a mold and solidifying the mixture in the mold. Said Japanese Patent suggests the fabrication of transdermal microneedles and the transdermal delivery of drugs through the fabricated microneedles, but the skin penetration of the microneedles involves pain. Due to the technical limitation in the fabrication of a mold, it is impossible to fabricate a microneedle, which has the length required for effective drug delivery, that is, a length of 1 mm or more, and, at the same time, an appropriate upper end diameter which causes no pain. For this reason, it is limited in its ability to allow a drug or a beauty component to permeate deep into the skin. Meanwhile, Prausnitz of the University of Georgia suggested a method of fabricating biodegradable polymer microneedles, which comprises producing a mold with glass by etching or photolithography, adding a biodegradable polymer to the mold, and solidifying the polymer in the mold (Biodegradable polymer microneedles: Fabrication, mechanics and transdermal drug delivery, Journal of Controlled Release 104, 2005, 5166 and Polymer Microneedles for Controlled-Release Drug Delivery, Pharmaceutical Research, Vol. 23, No. 5, May 2006 1008). In the fabrication of such transdermal biodegradable microneedles, the fabrication of the mold for forming the external shape of the microneedles should come first, and the deformation and loss of the external shape occur in a process of separating the microneedles from the mold.

Since the biodegradable solid microneedles are not removed from the body after their insertion into the body, they should cause minimal pain when they penetrate the skin, give less foreign body sensation after their insertion into the body, and, at the same time, have such a hardness that they be effectively delivered to the target site via the stratum corneum. The skin is comprised of the stratum corneum (<20 μm), the epidermis (<100 μm) and the dermis (100-3,000 μm). Thus, in order to deliver drug or skin cosmetic components to all the layers of the skin or a certain skin layer, the microneedles are preferably fabricated to have an upper end diameter of 5-40 μm and an effective length of 1,000-2,000 μm. Furthermore, such biodegradable solid microneedles should be able to be fabricated using a drug or a cosmetic component as a raw material. In the prior solid microneedles, the raw material thereof was limited to materials such as silicon, polymers, metal, glass or the like, due to the limitation on the fabrication methods thereof, and it was not easy to achieve the desired effects, because they were fabricated to have a diameter of 50-100 μm at the upper end part and a length of 500 μm.

Therefore, there has been a continued need for microneedles, which have a diameter small enough to realize painless penetration into the skin, and a length long enough to penetrate deep into the skin, and, at the same time, have sufficient hardness without any particular limitation on the raw materials thereof, as well as a fabrication method thereof.

### DISCLOSURE

### Technical Problem

Accordingly, the present inventors have made a great effort to develop a novel method for fabricating microneedles and, as a result, found that drawing lithography overcomes the limitation of the prior art, thereby completing the present invention.

Therefore, it is an object of the present invention to provide solid microneedles.

Another object of the present invention is to provide a method for fabricating solid microneedles.

### Technical Solution

To achieve the above objects, the present invention provides a method of using drawing lithography to fabricate biodegradable solid microneedles. According to the present invention, the entire surface of a substance is first coated with a biodegradable viscous material to be formed into microneedles. Alternatively, only the portion of the substrate, on which microneedles are to be formed, that is, the area that is to be brought into contact with pillars formed on a frame in the desired pattern, is selectively coated with the polymer to form a pattern. The coated material is maintained at a suitable temperature, such that it is not solidified. After the pillars formed on the frame in the desired pattern are brought into contact with the surface of the coated viscous material, the coated viscous material is solidified while it is drawn with the frame. As a result, the coated viscous material forms a structure which has a diameter decreasing from the substrate toward the surface contacting with the frame. The drawing process can be carried out by fixing the substrate and moving the frame upward or downward. Alternatively, it can also be performed by fixing the frame and moving the substrate upward or downward. At this time, biodegradable solid microneedles having a thin and long structure are fabricated either by increasing the drawing speed, such that a force greater than the tensile strength of the coated material is applied to the coated material, or by cutting a specific portion of the coated material using a laser beam. In the present invention, drawing temperature and drawing speed are suitably controlled depending on the properties of the coated material, for example, viscosity, and the desired structure of the biodegradable solid microneedles. In summary, the method for fabricating biodegradable solid microneedles according to the present invention comprises the steps of: i) coating the surface of a substrate with a viscous material for forming biodegradable solid microneedles; ii) bringing the surface of a frame having pillar patterns formed thereon, into contact with the surface of the coated viscous material; iii) drawing the coated viscous material using the frame, while solidifying the viscous material; and iv) cutting the drawn material at a given position thereof, thus obtaining biodegradable solid microneedles.

In the present invention, the viscous material that is used to form the biodegradable solid microneedles is not specifically limited. For example, various materials, such as hydrogel, maltose, drugs for the treatment for skin diseases, cosmetic components, water-soluble materials and polymeric proteins, may be used to form the biodegradable solid microneedles.

In the present invention, the number of the pillar patterns of the frame is not specifically limited, and a large number of pillar patterns may be used to produce a large amount of microneedles.

In the present invention, the cutting of the microneedles can be performed by increasing the drawing speed or applying to the material a force greater than the tensile strength of the material, but the scope of the present invention is not limited thereto.

It is important that microneedles should have a structure, which is thin and long enough to minimize not only pain in their penetration into the skin, but also foreign matter sensation after their insertion into the skin. According to the present invention, the solid microneedles can be fabricated to have the desired diameter and length without any particular limitation. Preferably, the solid microneedles can be fabricated to have an upper end diameter of 5-40 μm and an effective length of 500-2,000 μm.

As used herein, the term “upper end” of microneedles means one end of the microneedle, at which the diameter is the minimum.

As used herein, the term “effective length” means the vertical length from the upper end of the microneedle to the position having a diameter of 50 μm. As used herein, the term “solid type microneedle” means a microneedle which is formed in the solid state without hollow holes.

As used herein, the term “biodegradable” means that in-vivo degradation occurs.

### DESCRIPTION OF DRAWINGS

FIG. 1 shows a frame and pillars patterned thereon, which are used for the drawing of microneedles.

FIGS. 2a to 2f schematically show the process of fabricating biodegradable solid microneedles according to the present invention.

FIGS. 3a to 3c show the structure of biodegradable solid microneedles according to the present invention.

FIGS. 4a to 4c show the structure of an array of the inventive biodegradable solid microneedles, fabricated in the form of a patch.

FIGS. 5a to 5d show a process in which an array of the inventive biodegradable solid microneedles, fabricated in the form of a patch, is applied to the skin. FIGS. 6a to 6d show a process in which an array of the inventive biodegradable solid microneedles, fabricated in the form of a patch, is applied to the skin.

FIG. 7 shows an example in which an array of the inventive biodegradable solid microneedles, fabricated in the form of a roller-type patch, is applied to the skin.

### BEST MODE

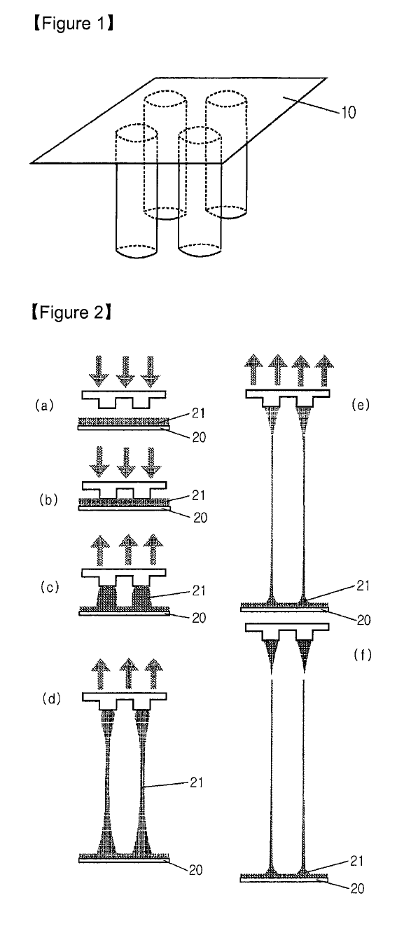

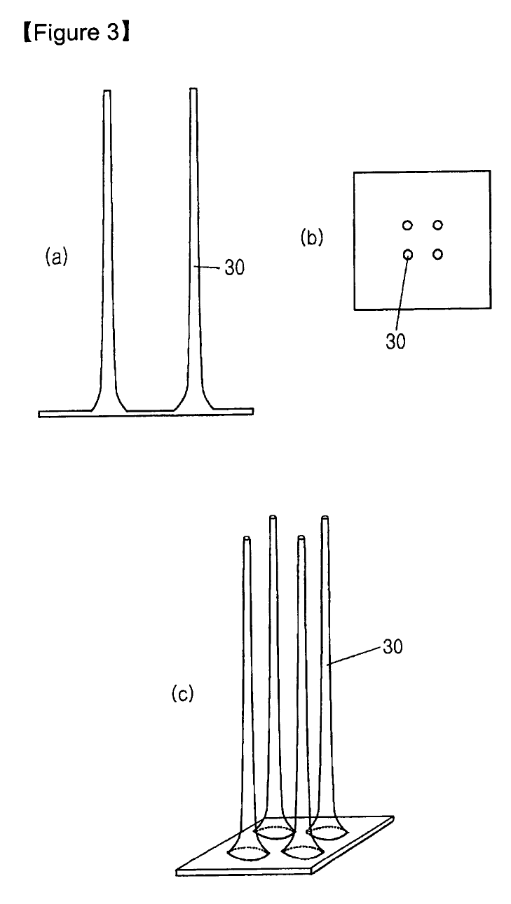

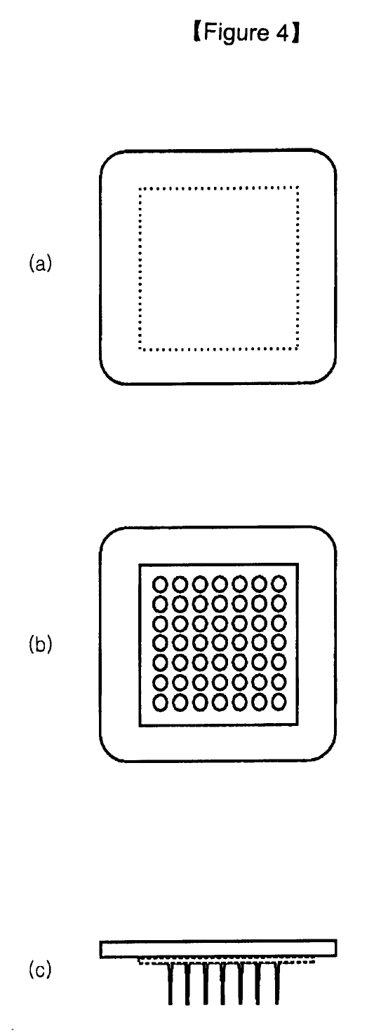

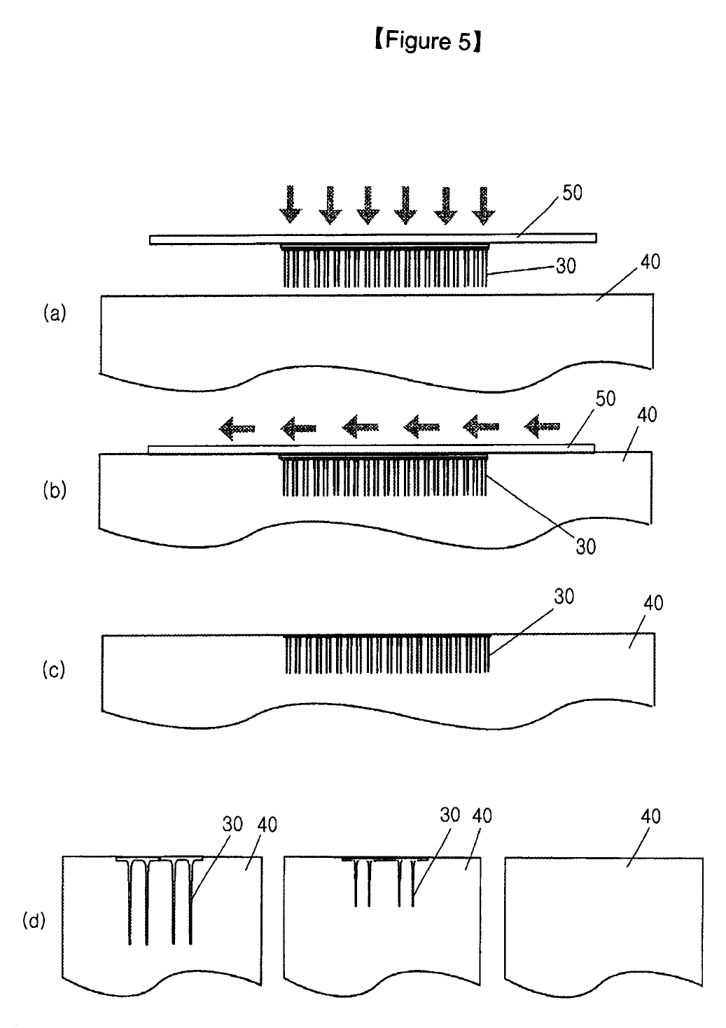

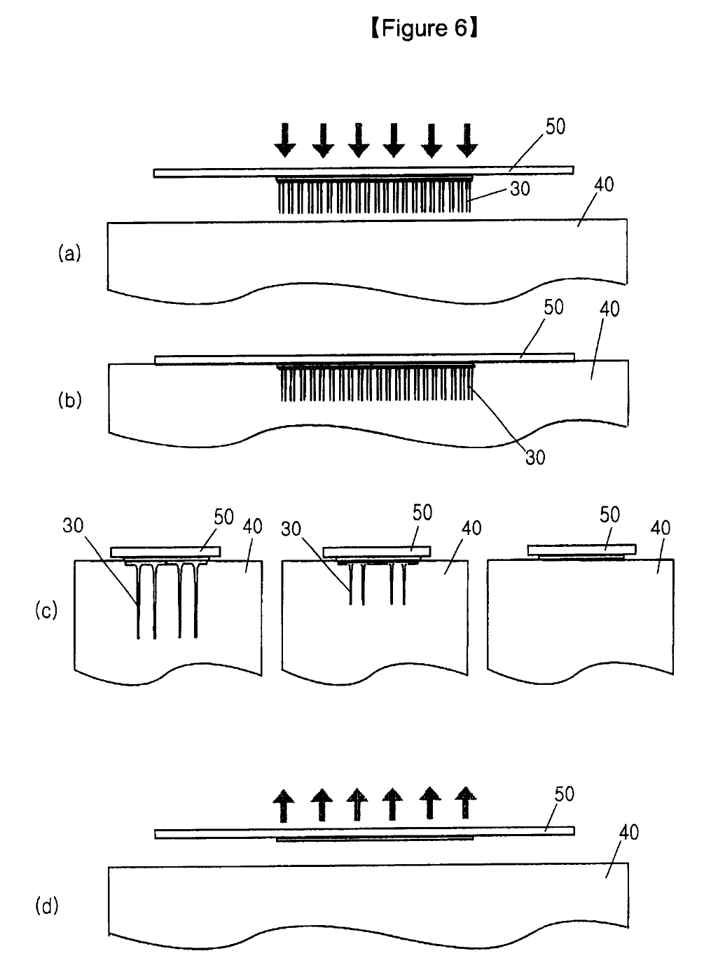

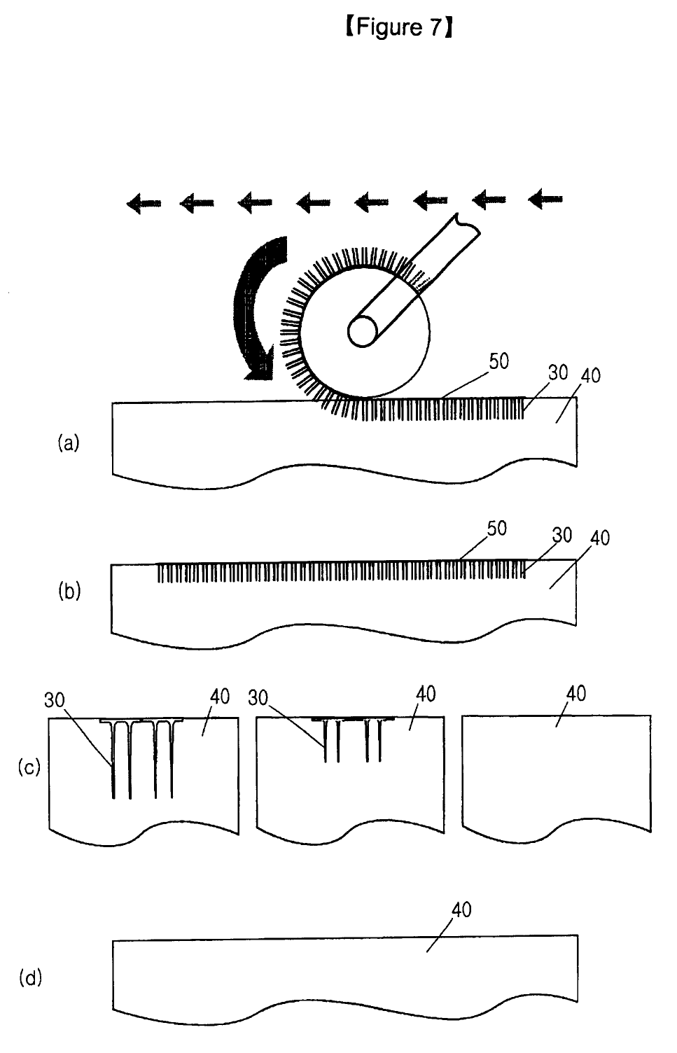

Hereinafter, the present invention will be described in further detail with reference to the accompanying drawings. FIG. 1 shows a frame 10 and 2×2 pillar patterns 20 formed thereon. Although the diameter of the resulting microneedles depends on the diameter of the pillar patterns formed on the frame, the diameter of the biodegradable solid microneedles may be made smaller than the diameter of the pillars patterned on the frame. Also, when a large number of pillar patterns are formed on the frame, it is possible to produce a large amount of microneedles. The frame is preferably made of one selected from among metals and reinforced plastics, which do not show a great change in their properties upon changes in temperature and humidity, but the scope of the present invention is not limited thereto. The frame used in the fabrication of the microneedles may be reused after washing. FIGS. 2a to 2f are views showing a process of fabricating solid microneedles. As shown in the figures, a parafilm, an aluminum foil or a band is first applied on a substrate 20 having excellent heat conductivity, such as glass or metal, and then a material for forming microneedles is coated on the substrate to form a film 21. The coated material, drawing rate and applied temperature are the main factors to decide the structure of the resulting biodegradable microneedles, and these factors may be suitably adjusted depending on the desired length and diameter. FIG. 3a is a side view of biodegradable solid microneedles 30 fabricated according to the method of the present invention; FIG. 3b is a plan view of the biodegradable solid microneedles 30; and FIG. 3c is a side view thereof, inclined at an angle of 45°. FIGS. 4a to 4c show biodegradable solid microneedles fabricated using an in-vivo absorbing material according to the present invention. FIGS. 5a to 5d and FIGS. 6a to 6d show an example where a patch 50 having the biodegradable solid microneedles 30 attached thereto is applied to the skin 40. Specifically, FIGS. 5a to 5d show that the patch 50 is removed immediately after it is used to insert the biodegradable solid microneedles 30 into the skin, and FIGS. 6a to 6d show that the patch 50 is removed after the biodegradable solid microneedles 30 inserted into the skin 40 are sufficiently absorbed into the skin 40. Meanwhile, FIGS. 7a to 7d show an example where the biodegradable solid microneedles 30 fabricated according to the present invention are applied to the skin 40 using a roller-type patch 50.

Hereinafter, the present invention will be described in further detail with reference to examples. It is to be understood, however, that these examples are illustrative only, and the scope of the present invention is not limited thereto. Also, it is to be understood that various modifications, variations or changes, which are apparent to one skilled in the art when reading the specification of the present invention, all fall within the scope of the present invention. All the literature cited in the present specification is incorporated herein by reference.

### EXAMPLES

SU-8 2050 photoresist (commercially purchased from Microchem) having a viscosity of 14,000 cSt was used to fabricate solid microneedles. For this purpose, SU-8 2050 was coated on a flat glass panel to a certain thickness, and it was maintained at 120° C. for 5 minutes to maintain its flowing properties. Then, the coated material was brought into contact with a frame having 2×2 pillar patterns formed thereon, each pillar having a diameter of 200 μm (See FIG. 1). The temperature of the glass panel was slowly lowered to 90-95° C. over about 5 minutes to solidify the coated SU-8 2050 and to increase the adhesion between the frame and the SU-8. Then, while the temperature was slowly lowered from 90-95° C., the coated SU-82050 was drawn at the speed of 1 μm/s for 60 minutes using the frame which adhered to the coated SU-82050 (See FIG. 2). After 60 minutes of drawing, solid microneedles, each having a length of about 3,600 μm, were formed. Subsequently, the solid microneedles were cured for 30 minutes, and then the drawing speed was increased to 700 μm/s in order to separate the microneedles from the frame, thus fabricating microneedles, each having a length of more than 2,000 μm. Alternatively, the formed microneedles could be separated from the frame by cutting. As a result, microneedles, each having an upper end diameter of 5-30 μm, an effective length of 2,000 μm and a total length of 3,000 μm, were fabricated.

In another Example, biodegradable plastic PLA (Poly-L-lactide (commercially available from Sigma) was used to fabricate biodegradable solid microneedles. Specifically, PLA was dissolved in dichloromethane (purchased from Sigma) as a solvent, and then PLA solution was coated on a flat glass panel to a given thickness. A frame having 2×2 pillar patterns formed therein, each pattern having a diameter of 200 μm, was brought into contact with the coated PLA solution. Due to the strong volatility of dichloromethane, the coated PLA solution was hardened, while the adhesion between the frame and the PLA solution was increased. After 3 minutes, the coated PLA was drawn at a speed of 25 μm/s for 90 seconds using the flame which adhered to the PLA solution, thus forming solid microneedles, each having a length of 2,200 μm. Subsequently, the formed solid microneedles could be separated from the frame by increasing the drawing speed or cutting the microneedles. Then, the separated biodegradable solid microneedles were crystallized in a vacuum oven at 170° C., thus obtaining biodegradable plastic microneedles, each having an upper end diameter of 5 μm, an effective length of 2,000 μm and a strength of 1.5 N.

In still another Example, carboxymethyl cellulose (CMC: purchased from Sigma), which is a cellulose derivative, was used to fabricate biodegradable microneedles. Specifically, CMC was dissolved in water as a solvent to make a 4% CMC solution. The CMC solution was coated on a flat glass panel to a given thickness and brought into contact with a frame having 2×2 pillar patterns formed thereon, each pillar having a diameter of 200 μm. For 10 seconds after the contact process, the coated CMC layer was dried to increase the adhesion between the frame and the CMC layer. The coated CMC was drawn at a speed of 30 μm/s for 60 seconds using the frame which adhered to the CMC, thus forming solid microneedles, each having a length of 1,800 μm. Subsequently, the microneedles were dried and solidified for 5 minutes, and the solidified microneedles could be separated from the frame by increasing the drawing speed or cutting the microneedles. As a result, biodegradable cellulose microneedles, each having an upper end diameter of 5 μm and an effective length of 1,800 μm, were fabricated.

In yet another Example, maltose monohydrate (purchased from Sigma), which is natural sugar, was used to fabricate biodegradable microneedles. Specifically, maltose monohydrate was melted at 140° C. to make a viscous maltose solution, which was then coated on a flat glass panel to a given thickness. Then, a frame having 2×2 pillar patterns formed thereon, each pillar having a diameter of 200 μm, was brought in contact with the coated maltose layer. For 10 seconds after the contact process, the adhesion between the coated maltose layer and the frame was increased. Then, the coated maltose was drawn at a speed of 30 μm/s for 60 seconds using the frame which adhered to the coated maltose layer, thus forming biodegradable solid microneedles, each having a diameter of 1,800 μm. Then, the solid microneedles were hardened for about 20 minutes, until the coated maltose reached 50° C. Subsequently, the formed biodegradable solid microneedles could be separated from the frame by increasing the drawing speed or cutting the microneedles. As a result, biodegradable maltose microneedles, each having an upper end diameter of 5 μm and an effective length of 1,800 μm, were fabricated. As described above, according to the present invention, it is possible to fabricate microneedles having a structure, which could not be achieved by the prior art. The solid microneedles having a diameter of less than 50 μm and a length of at least 1 mm, fabricated according to the present invention, will be useful for the in-vivo delivery of not only drugs or beauty components, but also polymer materials or water-soluble materials, which were difficult to deliver in-vivo in the prior art.

1. A method for fabricating microneedles, the method comprising:
coating a surface of a substance with a biodegradable viscous material to form microneedles;
drawing the coated biodegradable viscous material using a frame having pillar patterns formed thereon while the biodegradable viscous material is being solidified; and
cutting the drawn biodegradable viscous material at a given position thereof.

2. The method of claim 1, wherein the viscous material is one selected from the group consisting of photoresist, biodegradable plastics, cellulose derivatives, maltose, and a combination thereof.

3. A microneedle fabricated according to the method of claim 2.

4. The microneedle of claim 3, which has an upper end diameter of 5-40 μm and an effective length of 500-2,000 μm.

5. A microneedle fabricated according to the method of claim 1.

6. The microneedle of claim 5, which has an upper end diameter of 5-40 μm and an effective length of 500-2,000 μm.
